(19) United States
(12) Patent Application Publication
(10) Pub. No.: US 2005/0265523 A1
(43) Pub. Date: Dec. 1, 2005

(54) C-arm device with adjustable detector offset for cone beam imaging involving partial circle scan trajectories

(76) Inventor: Norbert Karl Strobel, Palo Alto, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/140,225

(22) Filed: May 27, 2005

Related U.S. Application Data
(60) Provisional application No. 60/575,193, filed on May 28, 2004.

(51) Int. Cl. H05G 1/02; A61B 6/00; G01N 23/00

(52) U.S. Cl. 378/193

(57) ABSTRACT

Method and system of generating a three dimensional reconstruction of a volume of a patient with an C-arm X-ray imaging system. More particularly, the method and system taught corrects for truncation projection errors by creating an effective detector of greater width.

### BACKGROUND OF THE INVENTION

1. Field of the Invention

The present invention generally relates to C-arm X-ray system used for medical imaging. In particular, the present invention relates to a novel method and system for the correction of truncated projections that occur in such C-arm X-ay systems.

2. Description of the Background Art

C-arm X-ray systems are currently used in medical imaging to create both 2-D and 3-D images (reconstructions). These systems Many C-arm X-ray systems, like other practical cone-beam imaging devices, are equipped with detectors that are to small to fully capture a projection of a given object. Such detectors cause truncated projections when recording views of objects that extend beyond the detector boundaries. Since the Detector Field of View (DFOV) determines the Scan Field of View (SFOV) a small detector limits the overall size of an object that can be examined and reconstructed without artifacts.

Mathematical extrapolation methods do exist to reduce the impact of truncated projections. However, for best results with these methods, a few views are required to have captured the overall object mass and the center of mass. Therefore, in these views the detector most cover the full projection of the object. Given the detector size and the object to be imaged, this is often not practical to achieve.

In addition to mathematical extrapolation methods, a variety of hardware modifications to X-ray systems have been proposed to address the problem of truncated projections. U.S. Pat. No. 5,032,990 to Eberhard et al. discloses a two position data acquisition scheme where an objected is translated and rotated relative to a stationary source-detector configuration. U.S. Pat. No. 5,740,224 to Muller et al. discloses a linear and circular synthetic scanner arrays where the scanner remain stationary and the object to be scanner is mounted on a turntable that can be displaced and rotated. Both of these proposed solutions are not easily applicable to a C-arm X-ray system.

Cho et al. has disclosed in the literature performing a full circle scan with a laterally offset detector. While this method increase the effective detector width, it is not applicable to C-arm X-ray systems as they can not perform complete circle scans but rather only partial circle scans.

Therefore, there exists a need in the art for a method or system to reduce or eliminate the problem of truncated projections in C-arm X-ray systems.

### SUMMARY OF THE INVENTION

The present invention solves the existing need according to a first aspect by providing a c-arm x-rays imaging system which has gantry, a c-arm, an x-ray source, and an x-ray detector. Further the x-ray detector can translate its center stage in the plane of the detector.

According to another aspect of the invention, a method is provided for taking two partial circle scans with center stage of the detector is opposite offset positions creating an effective detector of larger size to avoid the problems of truncated projections.

According to yet another aspect of the invention, a method is provided for is provided to generating calibration data including projection matrices and offset transform parameters need to generate the projection matrices for the partial circle scans of opposite offset central stage of detectors.

### BRIEF DESCRIPTION OF THE DRAWINGS

The invention will become more clearly understood from the following detailed description in connection with the accompanying drawings, in which:

FIG. 1 is a view of an C-arm x-ray imaging system.

FIG. 2 is a view of a detector with a movable center stage.

FIG. 3 is a view of a cross-roller bearing.

FIG. 4 is a flow chart of method needed to avoid truncation projection errors.

FIG. 5 is a flow chart of a calibration method.

FIG. 6 is the data image of a calibration phantom.

### DETAILED DESCRIPTION OF THE PREFERRED EMBODIMENTS

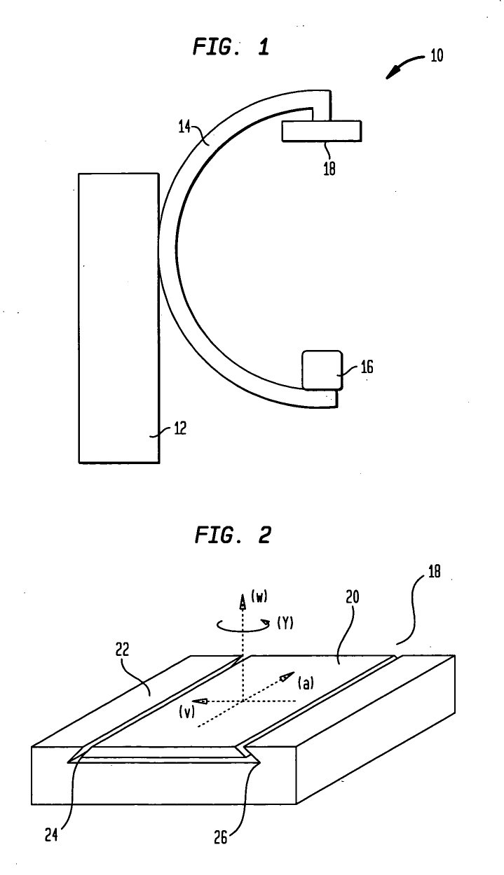

Referring to FIG. 1, a C-arm X-ray imaging system 10, having a gantry 12 supporting a C-arm 14. The C-arm 14 has atone end an X-ray source 16 and a detector 18 at the other end. The C-arm 14 defines a plane. The C-arm nay swivel in the around an axis perpendicular to the pane in process called angulation. The C-arm 14 may also swivel around an axis perpendicular to the pane in orbital rotation. During a partial circle scan, the C-arm 14 will angulate to generate views from multiple angles. The detector 18 itself rotates around the axes defined by the detector 18 and the source 16.

In an one embodiment of the present invention, the detector 18 may be a free bilateral offset detector as shown in FIG. 2. The detector 18 has a central stage 20 and a detector mount 22. The detector mount 22 includes slides 24 and 26 to hold and translate the central stage 30. The slides 24 and 26 may be dove tails other structures well known to the mechanical arts.

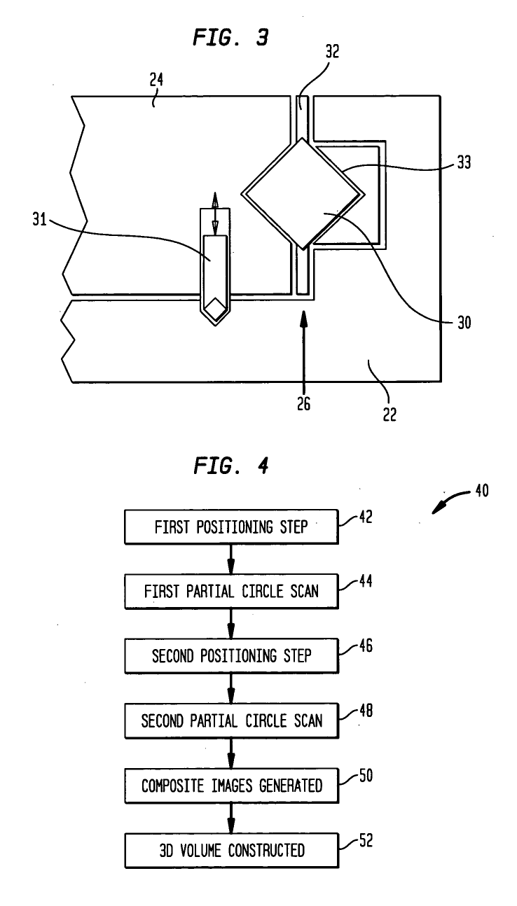

In one embodiment of the present invention the detector 18 may have slides 24 and 26 having cross cross-roller bearings. Such cross-roller bearings avoid the problems of friction and striction present in dovetail joints. FIG. 3 shows a cross-roller bearing 30 having a clamping pin 31, a cage 32, a preload 33. The clamping pin 30 is a way of fixing the lateral movement of the central stage 20 in s specific and reproducible position. However, if cross-roller bearing the central stage 20 position could be precisely determined at all times, then such a clamping mechanism would be unnecessary.

One embodiment of the present invention is a method of using imaging system 10 the imaging system as shown in FIG. 4. The center stage 20 is positioned in a first position in step 42 by moving it by a lateral offset ΔL from the center. A first partial circle scan 44 is performed with the center stage 20 in the first position. The center stage 20 is then shifted to a second position −ΔL from the center in step 46. A second partial scan in step 48. In step 50 a composite view is synthesized by interpolation. In step 52 Feldkamp or other reconstruction algorithms are applied to reconstruct a 3D volume based on the reconstructed views.

The process above thus creates two sets of partial scans a defined displacement from each other with the same source cone of X-rays. This allows to the creation of an effective detector with eliminates the truncation projections.

In order to make this method effective, the projection geometry most be determined by calibration. A calibration phantom is typically placed at the C-arm iso-center, where the calibration phantom is completely scene for every view.

If the detector offset is small, for example ΔL=10 cm where the center stage is 40 cm, the standard calibration procedure will suffice, with the modification that it be run twice, once for each position of the central stage of the detector.

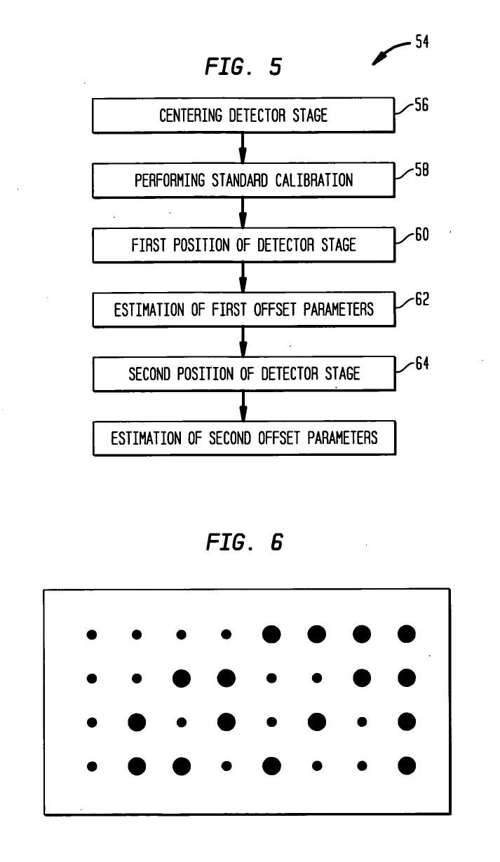

However, if the detector offset is a large amount, each view will not capture the full projection of the calibration phantom. FIG. 5 shows the relevant procedure for calibration 54. In step 56 the center stage of the detector is centered with regard to the detector mount. In step 58 a standard calibration is performed with the central stage of the detector centered. This generates a projection matrix. In step 60 the central stage of the detector is placed in a first offset position. In step 62 the offset parameters are estimated for that fist position. In step 64 the center stage of the detector is set to a second position oppositely offset for the first position. In step 66 the offset parameters are estimated for the second position.

The result of the above procedure is a projection matrix for the centered detector, and a offset parameters for the offset position. The final projection matrices used for each actual partial circle scan can either be generated off line, or the centered projection matrix can be stored with the offset parameters, and the appropriate projection matrices can calculated “on-line” during the scan. This second approach has the advantage of using one calibration for the centered matrix, and then storing a number of different offset parameters to allow for different (standard) offset of the central stage of the detectors. For example, different organs may require different detector offsets to avoid truncation errors due to the size of the organs. Thus, for a specific organ a specific offset can be used, with the offset parameters for that position stored and ready to be used.

In a particular embodiment of the present invention, the above described projection matrix and offset (transform) parameters are related as described below. The projection geometry of the n^{th} view with the projection matrix Pₙ is for N viewing positions (projection angles). A projection is taken under Pₙ when we mane that is taken with the source in its n^{th} position.

Assuming a very precise mechanical shift mechanism that restricts the shift to be (mostly) planar and a clamp fixing the detector such that it cannot move during C-arm rotation, the shift parameters may be estimated under one particular C-arm viewing angle along the image acquisition trajectory, e.g. the posterior-anterior position. If the detector cannot be rigidly fixed in its offset positions, we have to estimate the shift/transform parameters for all N viewing positions.

Assuming a stable clamping mechanism, the default projection matrix for the chosen view geometry is called P₀. The associated projection matrix with the detector at its position I^{th} shift position (to the right) is denoted P₀⁽ⁱ⁾. It can be computed from P₀ by taking P₀⁽ⁱ⁾=Tᵢ·Pₙ with a suitably chosen transform matrix Tᵢ. One possible choice to Tᵢ is a Eucliclean similarity transform (Eucliclean warp) defined as

$$Ti = [ \begin{matrix}{S}_{i} cos {α}_{i} ) & {s}_{i} sin ( {α}_{1} ) & {t}_{u}^{( i )} \\ - {s}_{i} sin ( {α}_{i} ) & {s}_{i} cos ( {α}_{i} ) & {t}_{v}^{( i )} \\ 0 & 0 & 1\end{matrix} ]$$

This transform involves four parameters for scale, Sᵢ, rotation, aᵢ, horizontal translation, tᵤ⁽ⁱ⁾, and vertical translation, tᵥ⁽ⁱ⁾. The transform matrix associated with Tᵢ, but with the detector shifted into its oppositely lateral position (to the left) is called T₋ᵢ.

To estimate the four parameters, at least two points that remain visible when projections are taken under P₀ and P₀⁽ⁱ⁾, respectively are needed. Once the shift parameters are estimated and assuming that a particular shift remains stable during the image acquisition run, the projection matrices are obtained for all other N−1 view directions according to Pₙ⁽ⁱ⁾=Tᵢ·Pₙ.

A simple calibration phantom facilitating the estimate of the shift parameters would be a Lucite plate embedded beads of two different sizes. If the beads are used to establish binary code words, the sizes must be chosen such that the larger beads are always significantly bigger than the smaller beads regardless of the magnification due to the divergent-beam projection geometry. Once beads of two significantly different sizes are provided, they can be used to express binary code words (e.g., a small bead for “0”, and a large bead for “1”). An interesting example is presented below. A linear code with 3 bits is used and one parity bit having a Hamming distance of two is used. In this case, neighboring columns always have two beads next to each other that have different size. In addition, each row has a unique pattern. Such a bead distribution makes it easier to pick (at least) four beads (two in each pair of adjacent columns) that are both seen under P₀ and P₀⁽ⁱ⁾, respectively. For a more reliable estimate of the transform parameters, more than two beads should be used. This may imply a different “code” design of the calibration plate. See FIG. 6

After two partial circle scans, the two sets of projects must be merged to create a composite projection. To combine the oppositely offset projections taken under Pₙ⁽ⁱ⁾=Tᵢ·Pₙ and Pₙ⁽⁻ⁱ⁾=T_{-I}·Pₙ define a new extended pixel grid that is associated with Pₙ. Then determine where the new grid positions are mapped onto the old grid positions. Old pixel grid positions on the detector shifted to the left are found by pre-multiplying the extended grid coordinates with T_{-I}⁻¹. If the oppositely shifted detectors have a center region in common, the associated gray levels in both projections are determined and then averaged. This way, noise is reduced, i.e., the fact that the overlapping detector region was irradiated twice is used. Clearly, from a dose usage point of view, keeping the overlap region small is preferred.

Due to the discrete nature of raster images, one is in no way assured that each pixel position in the extended grid maps to another (discrete) pixel position on the offset grid. In fact, the resulting gray level in the extended pixel grid should be determined by bi-linear interpolation between the neighboring samples of the old pixel grids.

After the composite create is create then standard 3D reconstruction techniques can be applied to image the volume being scanned.

The invention having been thus described, it will be apparent to those of skill in the art that the same may be varied in many ways without departing from the spirit and scope of the invention. Any and all such variations as would be apparent to those skilled in the art are intended to be covered by the following claims.

1. An x-ray imaging system comprising:
a) a gantry;
b) a C-arm mounted on the gantry;
c) an x-ray source mounted to one end of C-arm;
d) an X-ray detector mounted to the opposite end of the C-arm having a detector mount, a pair of slides, and a central stage held by said slides; and
e) wherein the central stage may translate along said guides parallel so said detector mount.

2. A imaging system of claim 1, wherein the detector is mounted to the C-arm such that the detector may rotate around the axis defined by the source and the detector.

3. A detector of an C-arm x-ray imaging system comprising:
a) a detector mount attached said C-arm;
b) a first and second slide parallel to each other;
c) a central stage mounted between said first and second slides wherein said central stage may translate along said slides.

4. The detector of claim 3, having a clamping pin and wherein the first and second slides each have one or more crossed roller bearings.

5. A method of imaging using a C-arm x-ray imaging system comprising the steps of:
a) positioning the center stage of a detector at a first position of lateral offset ΔL from the center.
b) performing a first partial circle scan to gather a first set of projection data;
c) positioning the center stage of a detector at a second position offset from the center of the detector by −ΔL; and
d) performing a second partial circle scan to gather projection data.

6. The method of claim 5, further comprising the steps of:
a) generating a composite projection data from the first and second sets of projection data; and
b) reconstruction of a volume from the composite projection data using a Feldkamp algorithm.

7. The method of claim 6, wherein performing a first and second partial circle scan includes generating first and second projection matrices with first and second transform parameters with a centered projection matrix.

8. A method of calibrating a C-arm x-ray imaging system comprising the steps of:
a) centering the central stage of the detector
b) performing a standard calibration;
c) generate a projection matrix from the standard calibration;
d) offsetting the central stage of the detector to a first position;
e) generating a first transform offset parameters;
f) offsetting the central stage of the detector to a second position; and
g) generating a second transform offset parameters.
